(19) United States
(12) Patent Application Publication
(10) Pub. No.: US 2021/0052335 A1
(43) Pub. Date: Feb. 25, 2021

(54) SURGICAL ROBOT ARM CONFIGURATION AND PLACEMENT

(71) Applicant: Verily Life Sciences LLC, South San Francisco, CA (US)

(72) Inventors: James Shuma, San Jose, CA (US); Joëlle Barral, Mountain View, CA (US); Michal Levin, Sunnyvale, CA (US)

(73) Assignee: Verily Life Sciences LLC, South San Francisco, CA (US)

(21) Appl. No.: 16/947,242

(22) Filed: Jul. 24, 2020

Related U.S. Application Data
(60) Provisional application No. 62/890,447, filed on Aug. 22, 2019.

(51) Int. Cl. A61B 34/30; A61B 90/92; A61B 90/50

(52) U.S. Cl. CPC A61B 34/30; A61B 2034/2057; A61B 90/50; A61B 90/92

(57) ABSTRACT

Systems and methods for setting up and evaluating positioning of robotic arms of a surgical device using procedure data including previous surgeries is described herein. Robotic surgical systems described herein include color-configurable lights connected to each robotic arm of the system. A first color and a second color are selected and emitted from each light and the color displayed by the light of each robotic arm is also displayed at the surgical console of the system. Further, systems and methods for providing a position quality score for each robotic arm and surgical port as well as the overall system are described. The position quality score is determined based on comparing the position of each robotic arm to a database of positions for the robotic arms during a procedure and lowering the score for deviations from the database of positions.

### CROSS-REFERENCE TO RELATED APPLICATIONS

This application claims priority to U.S. Provisional Patent Application No. 62/890,447, filed Aug. 22, 2019, titled “Surgical Robot Arm Configuration And Placement,” the entirety of which is hereby incorporated by reference.

### BACKGROUND

In recent years, robotic surgeries have become increasingly popular because of their advantages over the traditional human-operated open surgeries. Surgical tools used in robotic surgeries have improved levels of dexterity over a human surgeon. Surgical tools used in robotic surgeries are interchangeable and connected to robotic arms for control by a surgeon. These tools can provide the surgeon maximum range of motion and precision. In addition, high-definition cameras associated with the surgical tools can provide a better view of the operating site to the surgeon than are otherwise typically available. Further, the small size of the robotic surgical tools allows the surgeries to be done in a minimally invasive manner thereby causing less trauma to the patient's body.

### SUMMARY

Various examples are described including systems, methods, and devices relating to configuring surgical robots.

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions. One general aspect includes a robotic surgical system having a plurality of robotic arms, at least two robotic arms of the plurality of robotic arms each configured to couple with an interchangeable surgical tool at an end thereof and each of the plurality of robotic arms having a corresponding light emitting device. The robotic surgical system includes a camera positionable to capture images of the at least two robotic arms. The robotic surgical system also includes one or more processors; and one or more non-transitory computer-readable media including computer-executable instructions that, when executed by the one or more processors, cause the one or more processors to: receive, from the camera, an image that depicts the at least two robotic arms and provide a representation of the image for presentation at a display. The instructions further cause the processors to determine a first color of a first robotic arm of the at least two robotic arms based on a first arm characteristic associated with the first robotic arm and determine a second color of a second robotic arm of the at least two robotic arms based on a second arm characteristic associated with the second robotic arm. The instructions then cause the processors to cause the light emitting device corresponding to the first robotic arm to emit the first color and cause the light emitting device corresponding to the second robotic arm to emit the second color. The instructions further cause the processors to provide a first graphical interface element and a second graphical interface element for presentation at the display together with the image, the first graphical interface element associating the first robotic arm with the first color and the second graphical interface element associating the second robotic arm with the second color. Other embodiments of this aspect include corresponding computer systems, apparatus, and computer programs recorded on one or more computer storage devices, each configured to perform the actions of the methods.

Another example includes a computer-implemented method, including: receiving an image from a camera positioned to view at least a portion of a robotic surgical system, the image depicting at least two robotic arms of the robotic surgical system. The computer-implemented method also includes determining a first color for a first robotic arm of the at least two robotic arms based on a first arm characteristic associated with the first robotic arm and determining a second color for a second robotic arm of the at least two robotic arms based on a second arm characteristic associated with the second robotic arm. The computer-implemented method includes causing a first light emitting device connected to the first robotic arm to emit the first color and causing a second light emitting device connected to the second robotic arm to emit the second color. The computer-implemented method also includes generating a first graphical interface element and a second graphical interface element, the first graphical interface element identifying the first robotic arm using the first color and the second graphical interface element identifying the second robotic arm using the second color. The computer-implemented method further includes causing the image to be displayed and causing the first and second graphical interface elements to be displayed with the image. Other embodiments of this aspect include corresponding computer systems, apparatus, and computer programs recorded on one or more computer storage devices, each configured to perform the actions of the methods.

Another general aspect includes a robotic surgical system, having a plurality of robotic arms, at least two robotic arms of the plurality of robotic arms each configured to couple with a surgical tool at an end thereof and a camera positionable to capture images of the at least two robotic arms. The robotic surgical system also includes one or more processors; and one or more non-transitory computer-readable media including computer-executable instructions that, when executed by the one or more processors, cause the one or more processors to: receive kinematic data for the at least two robotic arms, the kinematic data corresponding to a position and an orientation of each of the at least two robotic arms. The instructions also cause the one or more processors to access orientation data corresponding to a plurality of reference positions and orientations for the plurality of robotic arms based on previous surgical procedures performed by robotic surgical systems, the orientation data representing positions and orientations of robotic arms of the robotic surgery systems during at least one of (i) initiation of a surgical procedure or (ii) while performing the surgical procedure. The instructions further cause the one or more processors to compare the kinematic data and the orientation data to identify one or more orientation differences for the at least two robotic arms and determine position quality scores for the at least two robotic arms based on the one or more orientation differences. The instructions further cause the one or more processors to provide, for presentation at a display, the position quality scores for the at least two robotic arms. Other embodiments of this aspect include corresponding computer systems, apparatus, and computer programs recorded on one or more computer storage devices, each configured to perform the actions of the methods.

Another general aspect includes a computer-implemented method, including: receiving kinematic data for at least two robotic arms of a robotic surgical system, the kinematic data corresponding to a position and an orientation of each of the at least two robotic arms. The computer-implemented method also includes accessing orientation data corresponding to a plurality of reference positions and orientations for the at least two robotic arms based on previous surgical procedures performed by robotic surgical systems, the orientation data representing positions and orientations of robotic arms of the robotic surgical systems during at least one of (i) initiation of the surgical procedure or (ii) while performing the surgical procedure. The computer-implemented method further includes comparing the kinematic data and the orientation data to identify one or more orientation differences for the at least two robotic arms and determining position quality scores for the at least two robotic arms based on the one or more orientation differences. The computer-implemented method also includes providing, for presentation at a display, the position quality scores for the at least two robotic arms. Other embodiments of this aspect include corresponding computer systems, apparatus, and computer programs recorded on one or more computer storage devices, each configured to perform the actions of the methods.

In yet another general aspect a computer-implemented method, including: receiving an image from a camera positioned to view at least a portion of a robotic surgical system, the image depicting at least two robotic arms of the robotic surgical system is described. The computer-implemented method also includes determining a first color for a first robotic arm of the at least two robotic arms based on a first arm characteristic associated with the first robotic arm and determining a second color for a second robotic arm of the at least two robotic arms based on a second arm characteristic associated with the second robotic arm. The computer-implemented method includes causing a first light emitting device connected to the first robotic arm to emit the first color and causing a second light emitting device connected to the second robotic arm to emit the second color. The computer-implemented method further includes receiving kinematic data for the at least two robotic arms, the kinematic data corresponding to a position and an orientation of each of the at least two robotic surgery. The computer-implemented method also includes accessing orientation data corresponding to a plurality of reference positions and orientations for the at least two robotic arms based on previous surgical procedures performed by robotic surgical systems, the orientation data representing positions and orientations of robotic arms of the robotic surgical systems during at least one of (i) initiation of the surgical procedure or (ii) while performing the surgical procedure. The computer-implemented method further includes comparing the kinematic data and the orientation data to identify one or more orientation differences for the at least two robotic arms and determining position quality scores for the at least two robotic arms based on the one or more orientation differences. The computer-implemented method also includes generating a first graphical interface element and a second graphical interface element, the first graphical interface element identifying the first robotic arm using the first color and including a first position quality score of the position quality scores corresponding to the first robotic arm, the second graphical interface element identifying the second robotic arm using the second color and including a second position quality score of the position quality scores corresponding to the second robotic arm. The computer-implemented method also includes causing the image to be displayed and causing the first and second graphical interface elements to be displayed with the image. Other embodiments of this aspect include corresponding computer systems, apparatus, and computer programs recorded on one or more computer storage devices, each configured to perform the actions of the methods.

### BRIEF DESCRIPTION OF THE DRAWINGS

The accompanying drawings, which are incorporated into and constitute a part of this specification, illustrate one or more certain examples and, together with the description of the example, serve to explain the principles and implementations of the certain examples.

FIG. 1 illustrates a block diagram illustrating an example system for configuring a surgical robot, according to at least one example.

FIG. 2 illustrates an example system for configuring a surgical robot, according to at least one example.

FIG. 3 illustrates a simplified block diagram depicting an example architecture for implementing the techniques described herein, according to at least one example.

FIG. 4 illustrates a simplified block diagram depicting elements for performing setup configuration of a surgical robot, according to at least one example.

FIG. 5 illustrates a simplified block diagram depicting elements for performing position quality determination of a surgical robot configuration, according to at least one example.

FIG. 6 illustrates an example user interface for presenting surgical robot configuration information, according to at least one example.

FIG. 7 illustrates an example flow chart depicting an example process for configuring a surgical robot, according to at least one example.

FIG. 8 illustrates an example flow chart depicting an example process for determining a position quality score, according to at least one example.

FIG. 9 illustrates an example flow chart depicting an example process for configuring a surgical robot and determining a position quality score for the configuration, according to at least one example.

### DETAILED DESCRIPTION

Examples are described herein in the context of configuring surgical robots for surgical procedures. Those of ordinary skill in the art will realize that the following description is illustrative only and is not intended to be in any way limiting. For example, the techniques described herein may be used to initially configure a surgical robot or to provide evaluation of surgical robot configuration during a surgical procedure. Though examples and techniques are described with reference to surgical robot configurations, the methods and systems described herein may be implemented in other robotic systems such as robotic assembly systems as part of an assembly line for a product. Reference will now be made in detail to implementations of examples as illustrated in the accompanying drawings. The same reference indicators will be used throughout the drawings and the following description to refer to the same or like items.

In the interest of clarity, not all of the routine features of the examples described herein are shown and described. It will, of course, be appreciated that in the development of any such actual implementation, numerous implementation-specific decisions must be made in order to achieve the developer's specific goals, such as compliance with application- and business-related constraints, and that these specific goals will vary from one implementation to another and from one developer to another.

In an illustrative example, a robotic surgery system includes one or more robotic surgery arms each including one or more surgery tools. The robotic surgery system also includes a surgeon console for managing operation of the robotic arms and a computer system having modules loaded thereon for setting up a surgical device, including connecting surgical tools and positioning the robotic arms. A module determines a unique color to associate with each robotic arm. Upon making the determination, the module causes a light connected to each robotic arm to display the unique color. In addition, with each of the robotic arms uniquely identified with a color, and the colors displayed by the color-configurable lights, the module provides instructions to a user for connecting surgical tools to each of the robotic arms and positioning the robotic arms before beginning the surgery. In this manner, the system simplifies the setup process by uniquely and readily identifying each robotic arm without relying on the orientation of the system within a room or relative to a surgical console. For example, in previous systems, instructions to a user may identify a tool to connect to a robotic arm at one corner of the system, though the user may misinterpret the instruction and connect the surgical tool to a robotic arm on an opposite corner. This resulted in inconsistent configurations of systems and the present system aids faster and more accurate setup of surgical devices.

In an illustrative example, a surgical robot configuration system includes a surgical robot device and a setup module. The surgical robot device includes multiple surgical robot arms, each including a color-configurable light. The setup module aids setup and configuration of the surgical robot device including multiple surgical robot arms by uniquely identifying each surgical robot arm with the color-configurable light. The system also includes a display device which displays image data from a camera connected to an end of one of the surgical robot arms. The setup module determines a color for each of the surgical robot arms based on data from a database of previous surgical procedures. The setup module also causes the color-configurable light on each surgical robot arm to emit the color determined by the setup module. Additionally, the setup module causes the image data displayed at the display to display graphical interface elements identifying each surgical robot arm within the field of view of the camera as displayed on the display of the surgical console. The colors of the graphical interface elements are determined by the setup module and correspond to the colors of the color-configurable lights.

The surgical robot configuration system may also include a quality module. The quality module accesses arm position data describing the present kinematics of the surgical robot arms and compares it to kinematic data of surgical robot arms associated with previous surgical procedures. The quality module determines a position quality score for each of the surgical robot arms based on present kinematics as well as the kinematic data. The position quality score may also take into account the port locations on the patient for the robotic arms, the port location describing the sites on the patient where the robotic arms insert into the surgical area. The position quality score provides a numerical or other score describing how closely the present kinematics match the kinematic data. This position quality score may be used to set up or aid a user in setting up the surgical robotic system and guide the user to accurately position robotic arms for a procedure. The quality module also outputs the position quality score to the display. The quality module may determine the quality score during initial setup of the surgical robot device and may also determine quality scores during distinct steps of the surgical procedure.

This illustrative example is given to introduce the reader to the general subject matter discussed herein and the disclosure is not limited to this example. The following sections describe various additional non-limiting examples and techniques relating to configuring a surgical robot for a surgical procedure.

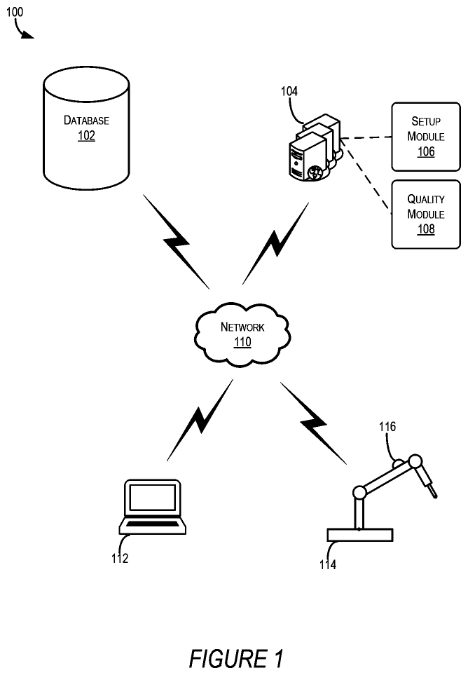

Turning now to the figures, FIG. 1 illustrates a block diagram of a system 100 for configuring a surgical device 114, according to at least one example. The system 100 includes computing device 104, surgical device 114, surgical console 112, and database 102. The surgical device 114 includes any suitable number of robotic arms. Each robotic arm of the surgical device 114 includes a color-configurable light 116 connected to the robotic arm. The computing device 104, database 102, and surgical console 112 may be in network communication with each other as shown through network 110. The setup module 106 communicates, via the network 110, with the database 102 and determines a unique identifier such as a color to be associated with each robotic arm of the surgical device 114. The setup module 106 further causes the color-configurable light 116 on each of the robotic arms to illuminate with the unique identifier color. The setup module 106 also causes the display of the surgical console 112 to include a graphical element identifying each robotic arm with the unique identifier color.

In some examples, the quality module 108 may communicate with the database 102 and the surgical device 114 to compare a position of each robotic arm to a stored position of the robotic arm from previous surgery data. Based on the comparison, the quality module 108 may generate a quality score indicating a quality of the positioning of the robotic arm. The quality module 108 may generate a quality score indicating a quality of the positioning of the ports for the robotic arms on the body of a patient as well. For example, the quality score may indicate how closely the robotic arm of the surgical device aligns with the stored position of the robotic arm or how closely the port locations match the stored port locations for the robotic arms. The quality module 108 then causes the quality score to be displayed on the surgical console 112 or otherwise presented to a surgeon and/or any other user.

The components of the system 100 are connected via one or more communication links with the network 110. The network 110 includes any suitable combination of wired, wireless, cellular, personal area, local area, enterprise, virtual, or other suitable network.

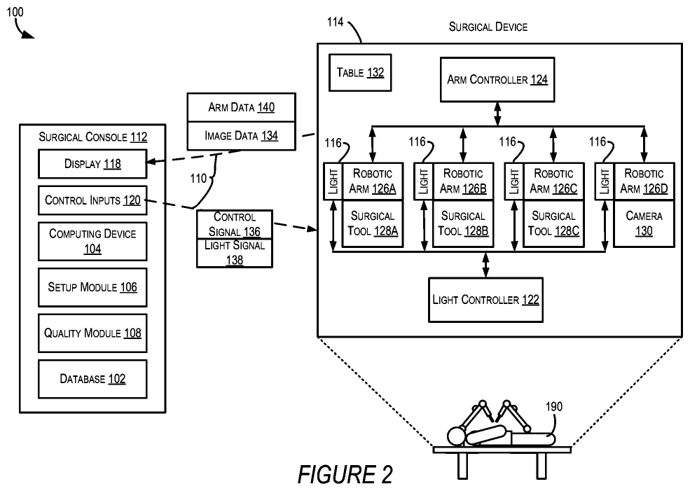

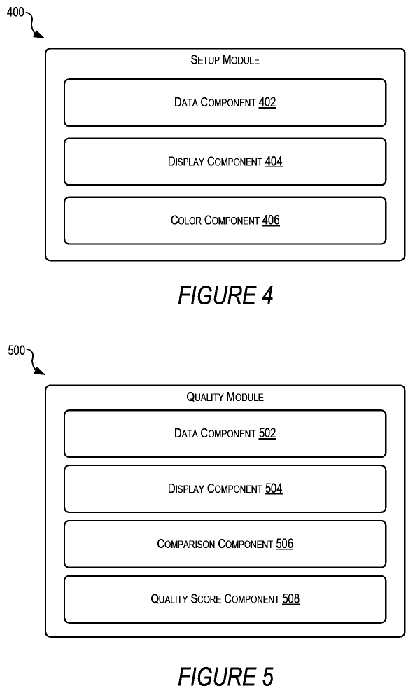

The computing device 104, as described herein, is any suitable electronic device (e.g., personal computer, hand-held device, server computer, server cluster, virtual computer, etc.) configured to execute computer-executable instructions to perform operations such as those described herein. As described in additional detail with respect to FIGS. 4 and 5, the computing device 104 includes a setup module 106 and a quality module 108, among other modules/components, and includes the functionality to perform the processes described herein. In some examples, the computing device 104 may be incorporated in or part of the surgical console 112. The surgical console 112 where a user controls the surgical device 114 and views the display 118 includes other components as described below with respect to FIG. 2.

It should be understood that although FIG. 1 illustrates the various components, such as the setup module 106, the quality module 108, and the database 102, that are included in the computing device 104 or in communication over the network 110, one or more of these modules may be implemented in different ways within the system 100. For example, the functionality described above need not be separated into discrete modules, or some or all of such functionality may be located on a computing device separate from the surgical device 114, surgical console 112, or computing device 104 such as a central controlling device connected to the surgical device 114 directly or through the network 110 and configured to control the components of the system 100.

FIG. 2 illustrates the system 100 for configuring surgical device 114, according to at least one example. In the system 100, surgical device 114 is configured to operate on a patient 190. The system 100 also includes a surgical console 112 connected to the surgical device 114 and configured to be operated by a surgeon to control and monitor the surgeries performed by the surgical device 114. The system 100 might include additional stations (not shown in FIG. 2) that can be used by other personnel in the operating room, for example, to view surgery information, video, etc., sent from the surgical device 114. The surgical device 114, the surgical console 112, and other stations can be connected directly or through the network 110, such as a local-area network (“LAN”), a wide-area network (“WAN”), the Internet, or any other networking topology known in the art that connects the surgical device 114, the surgical console 112 and other stations.

The surgical device 114 can be any suitable robotic system that can be used to perform surgical procedures on the patient 190. The surgical device 114 may have one or more robotic arms 126A-D (which may be referred to herein individually as a robotic arm 126 or collectively as the robotic arms 126) connected to a base such as a table 132. The robotic arms 126 may be manipulated by control inputs 120, which may include one or more user interface devices, such as joysticks, knobs, handles, or other rotatable or translatable devices to effect movement of one or more of the robotic arms 126. The robotic arms 126A-C may be equipped with one or more surgical tools 128A-C to perform aspects of a surgical procedure. For example, the robotic arms 126A-C may be equipped with surgical tools 128A-128C, (which may be referred to herein individually as a surgical tool 128 or collectively as the surgical tools 128). The surgical tools 128 can include, but are not limited to, tools for grasping for holding or retracting objects, such as forceps, graspers and retractors, tools for suturing and cutting, such as needle drivers, scalpels and scissors, and other tools that can be used during a surgery. Each of the surgical tools 128 can be controlled by the surgeon through the surgical console 112 including the control inputs 120.

Different surgical devices may be configured for particular types of surgeries, such as cardiovascular surgeries, gastrointestinal surgeries, gynecological surgeries, transplant surgeries, neurosurgeries, musculoskeletal surgeries, etc., while some may have multiple different uses. As a result, different types of surgical robots, including those without robotic arms, such as for endoscopy procedures, may be employed according to different examples. It should be understood that while only one surgical device 114 is depicted, any suitable number of surgical devices 114 may be employed within system 100.

The surgical device 114 is also equipped with one or more cameras 130, such as an endoscope camera, configured to provide a view of the operating site to guide the surgeon during the surgery. In some examples, the camera 130 can be attached to one of the robotic arms 126D. In some examples, the camera 130 can be attached to a mechanical structure of the surgical device 114 that is controlled separately from the robotic arms 126 or is stationary with respect to the surgical device 114.

The surgical device 114 includes an arm controller 124 as well as a light controller 122. The light controller 122 communicates with each of the color-configurable lights 116 connected to the robotic arms 126 based on a light signal 138 received from the surgical console 112. The arm controller 124 likewise controls the positioning and movement of the robotic arms 126 based on a control signal 136 from the surgical console 112 generated by the control inputs 120.

The surgical console 112 includes a display 118 for providing a feed of image data 134 from the camera 130. The image data 134 is transferred to the surgical console 112 over network 110 along with arm data 140 describing the position of each of the robotic arms 126. The computing device 104 described in FIG. 1 is shown included in the surgical console 112 but may also be located remotely of the surgical console 112 as described above.

During setup of the system 100, including connection of the surgical tools 128 and positioning of the robotic arms 126, the setup module 106 determines a unique color for each of the color-configurable lights 116. The unique color may be based on information from the database 102, or may be set based on user-specific preferences. For example, the setup module 106 may determine that first robotic arm 126A will be identified with the color blue, second robotic arm 126B will be identified with the color red, robotic arm 126C will be identified with the color green, and robotic arm 126D will be identified with the color yellow. Upon making the determination, the setup module 106 sends the light signal 138 to the light controller 122 instructing the light controller 122 to cause each of the color-configurable lights 116 to illuminate based on the colors previously selected. In addition, with each of the robotic arms 126 uniquely identified with a color, and the colors displayed by the color-configurable lights 116, the setup module 106 provides setup instructions to a user including a surgical tool 128 to connect to each of the robotic arms. With the robotic arms 126 each uniquely identified with an easy to identify marker, the instructions from the setup module 106 may instruct the user to connect a grasper to the blue robotic arm and other surgical tools 128 to the other robotic arms 126. The system 100 simplifies the setup process by uniquely and readily identifying each robotic arm 126 without relying on the orientation of the system 100 within a room or relative to the surgical console 112. For example, in previous systems, instructions to a user may identify a tool to connect to a robotic arm 126 at one corner of the system 100, though the user may misinterpret the instruction and connect the surgical tool to a robotic arm on an opposite corner. This results in inconsistent configurations of system 100 and may result in difficult or complex positioning of the robotic arms during the procedure. The techniques described herein of setting up the system 100 reduces the possibility of connecting surgical tools incorrectly or onto the wrong robotic arm.

The computing device 104 presents the image data 134 received from camera 130 on the display 118. The setup module 106 generates a graphical interface element to identify the unique color associated with each robotic arm 126 on the display 118 as portions of each robotic arm 126 are visible in the field of view of the camera 130 and also within the image data 134. The display 118 may not show the color-configurable light 116 on the display 118 but the user may wish to see each unique color identifying the robotic arms 126 on the display 118. In some instances, such as during a surgical procedure, a user may wish to identify each robotic arm 126 in the physical world with the unique color of each color-configurable light 116 as well as on the display 118 where the graphical interface element identifies each robotic arm 126 with the unique color. This may be especially useful during a surgical procedure but may also be helpful for a user at the initial setup of the surgical device 114 where the user may need to identify the robotic arms 126 in the physical world as well as identifying the end of the robotic arm 126 with the interchangeable tool on the display 118.

The quality module 108, which may be included on the computing device 104 in the surgical console 112, interfaces with the surgical device 114 to assist in preoperative positioning or intraoperative positioning of robotic arms 126, to compute quality scores, and to provide the quality scores for consumption at the display 118. The quality score indicates adherence or compliance with position data for each of the robotic arms 126 as stored in the database 102. Additionally, the quality score indicates adherence or how closely the location of surgical ports matches port locations stored in the database 102 to aid with setup of the surgical device 114. For example, the arm controller 124 may relay positioning instructions from the control inputs 120 to the robotic arms 126 and also returns arm data 140 describing the current position of each robotic arm 126. The quality module 108 accesses the data from the database 102 and compares the arm data to determine a quality score describing how nearly the arm data 140 and the data match. The quality score may be a numerical score, such as a score from 1-100, or may be a rating on any other scale to indicate the level of adherence. The data may include data describing robotic arm 126 positions in previous surgeries, averages of previous surgery data, or even predicted positions. The data may further be adjusted based on patient parameters as described below to normalize the arm data and the data to similar size scales based on patient parameters such as length, body mass index (BMI), weight, or other such physical parameters. The quality score is displayed on the display 118 and may include instructions for a user to adjust the position of the robotic arms 126 to increase the quality score.

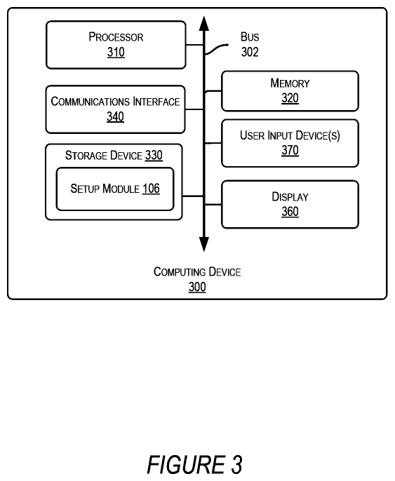

Referring now to FIG. 3, FIG. 3 shows computing device 300 suitable for use in example systems or methods for improving robotic surgical safety via video processing. For example, computing device 300 may be the computing device 104 of FIGS. 1 and 2.

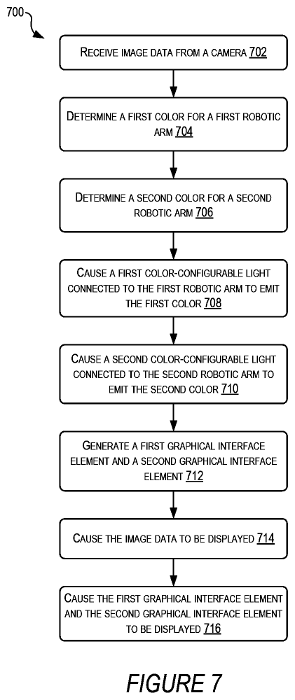

Computing device 300 includes a processor 310 which is in communication with the memory 320 and other components of the computing device 300 using one or more communications buses 302. The processor 310 is configured to execute processor-executable instructions stored in the memory 320 to perform security check of the surgical device 114 according to different examples, such as part or all of the example processes 700, 800, and 900 described below with respect to FIGS. 7, 8, and 9. The computing device 300, in this example, also includes one or more user input devices 370, such as a keyboard, mouse, touchscreen, microphone, etc., to accept user input. The computing device 300 also includes a 360 display to provide visual output to a user.

The computing device 300 can include or be connected to one or more storage devices 330 that provides non-volatile storage for the computing device 300. The storage devices 330 can store system or application programs and data used by the computing device 300, such as modules implementing the functionalities provided by the setup module 106 and the quality module 108. The storage devices 330 might also store other programs and data not specifically identified herein.

The computing device 300 also includes a communications interface 340. In some examples, the communications interface 340 may enable communications using one or more networks, including a local area network (“LAN”); wide area network (“WAN”), such as the Internet; metropolitan area network (“MAN”); point-to-point or peer-to-peer connection; etc. Communication with other devices may be accomplished using any suitable networking protocol. For example, one suitable networking protocol may include the Internet Protocol (“IP”), Transmission Control Protocol (“TCP”), User Datagram Protocol (“UDP”), or combinations thereof, such as TCP/IP or UDP/IP.

While some examples of methods and systems herein are described in terms of software executing on various machines, the methods and systems may also be implemented as specifically configured hardware, such as field-programmable gate array (FPGA) specifically to execute the various methods. For example, examples may be implemented in digital electronic circuitry, or in computer hardware, firmware, software, or in a combination thereof. In one example, a device may include a processor or processors. The processor comprises a computer-readable medium, such as a random access memory (RAM) coupled to the processor. The processor executes computer-executable program instructions stored in memory, such as executing one or more computer programs. Such processors may comprise a microprocessor, a digital signal processor (DSP), an application-specific integrated circuit (ASIC), field programmable gate arrays (FPGAs), and state machines. Such processors may further comprise programmable electronic devices such as PLCs, programmable interrupt controllers (PICs), programmable logic devices (PLDs), programmable read-only memories (PROMs), electronically programmable read-only memories (EPROMs or EEPROMs), or other similar devices.

Such processors may comprise, or may be in communication with, media, for example computer-readable storage media, that may store instructions that, when executed by the processor, can cause the processor to perform the steps described herein as carried out, or assisted, by a processor. Examples of computer-readable media may include, but are not limited to, an electronic, optical, magnetic, or other storage device capable of providing a processor, such as the processor in a web server, with computer-readable instructions. Other examples of media comprise, but are not limited to, a floppy disk, CD-ROM, magnetic disk, memory chip, ROM, RAM, ASIC, configured processor, all optical media, all magnetic tape or other magnetic media, or any other medium from which a computer processor can read. The processor, and the processing, described may be in one or more structures, and may be dispersed through one or more structures. The processor may comprise code for carrying out one or more of the methods (or parts of methods) described herein.

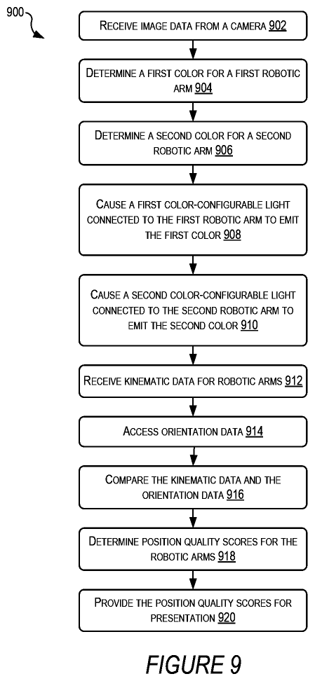

FIG. 4 illustrates a simplified block diagram depicting a setup module 400 with components for performing setup configuration of a surgical device 114, according to at least one example. The setup module 400 is an example of the setup module 106 described above with respect to FIGS. 1 and 2. The setup module 400 may include any suitable logical or physical divisions such as separate databases, memory modules, as well as suitable combinations of hardware, software, or firmware configured to implement the functionality of the methods described herein, such as the processes 700 and 900 described below with respect to FIGS. 7 and 9. To this end, the setup module 400 includes a data component 402, a display component 404, and a color component 406.

Turning now to the data component 402, the data component 402 is configured to interface with or serve as a database, such as database 102 to access data for use by the setup module 400 including setup configurations, color selections, user-specific preferences, and the like. The data component 402 stores information such as the data described above and is capable of selecting data for use by the setup module 400 based on procedure type, user identity, and the like.

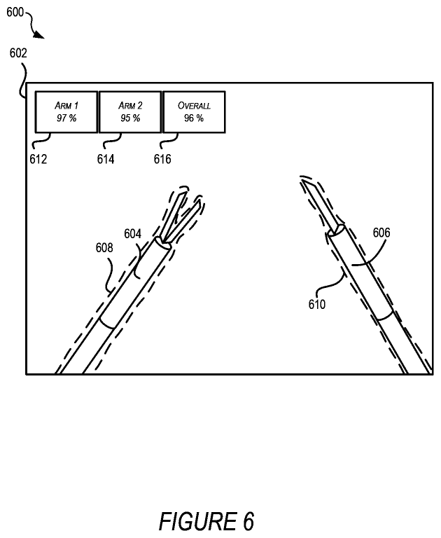

The display component 404 is configured to provide the image data 134 for display at the display 118 of the surgical console 112 as well as generate a graphical interface element to display with the image data 134. The graphical interface element is described in further detail with respect to FIG. 6 below. The display component 404 may be configured to augment the image data 134 and may also include components to perform object recognition within the image data to identify robotic arms 126. The display component may be configured to uniquely identify each robotic arm 126 based on the object recognition as well as the arm data 140.

Turning now to the color component 406, the color component 406 is configured to determine a unique color for each of the color-configurable lights 116 uniquely identifying each robotic arm 126. The color component 406 may be configured to interface with the data component 402 to use a set of colors based on surgeon preferences or based on data. For instance, a particular surgeon may prefer to have a grasper identified with the color blue in every surgery. Alternatively, it may be standard practice to identify the endoscope with the color green and a cutting tool with the color red as presented in the data accessed by the data component 402. The color component 406 is also configured to interface with the display component 404 to provide the unique colors for each robotic arm 126 for inclusion with the graphical interface element on the display 118.

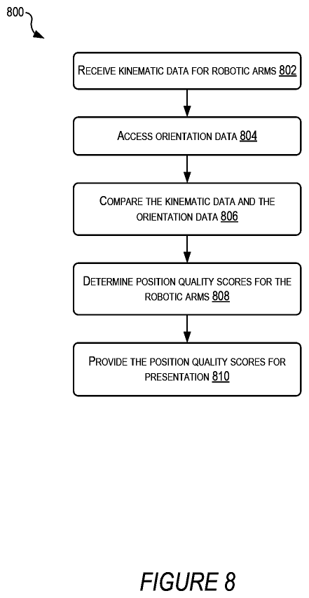

FIG. 5 illustrates a simplified block diagram depicting a quality module 500 with elements for performing position quality determination of a surgical robot configuration, according to at least one example. The quality module 500 is an example of the quality module 108 described above with respect to FIGS. 1 and 2. The quality module 500 may include any suitable logical or physical divisions such as separate databases, memory modules as well as suitable combinations of hardware, software, or firmware configured to implement the functionality of the methods described herein, such as the processes 800 and 900 described below with respect to FIGS. 8 and 9. To this end, the quality module 500 includes a data component 502, a display component 504, a comparison component 506, and a quality score component 508.

Turning now to the data component 502, the data component 502 is configured to interface with or serve as a database, such as database 102 to access data for use in the position quality score determination. The data component 502 stores information such as position data for each of the robotic arms 126 at the start of and throughout a procedure. The position data may include explicit positions of each robotic arm 126 and joint including joint angles as well as surgical port locations on the body of the patient. The position data may also include statistical distributions, such as averages or standard deviations for accepted positions of the robotic arms 126 and surgical ports. The data component 502 tracks the information and adds it to database 102. For example, the information may include new surgical procedure data such as sequences of robotic arm positions tracked and recorded during a recent procedure or surgeon specific preferences for positions of robotic arms during the procedure.

The comparison component 506 is configured to compare the arm data 140 describing the positions of the robotic arms 126 against the database 102 as well as the locations of the surgical ports against the locations stored in database 102. In some examples, the comparison component 506 is configured to receive a procedure input which narrows down the database 102 to a subset of data including similar procedures to the surgical procedure to be performed. For example, the procedure input may identify a surgical procedure and a procedure location, such as a surgery in the lungs for a lung problem such as lung cancer. Based on this identification, the comparison component 506 selects a subset of the database 102 covering procedures in the lungs and specifically for lung cancer procedures. In some examples the procedure input may further narrow down the dataset based on the particular location, such as the exact location within the lung. As the database 102 grows, further filters or procedure inputs may be used to narrow down the data from database 102 used in the comparisons and procedures described herein. The comparison component 506 may compare the position of each robotic arm 126 joint by joint against the data. In some examples, the comparison component 506 compares the position of each robotic arm 126 based on explicit positions and angles of the robotic arms. The comparison may also be based on statistical comparisons such as averages of robotic arm positions and joint angles or statistical distributions such as positions and angles within one standard deviation or other statistical ranges. In other examples, the comparison may be performed based on other statistical comparisons or mathematical comparisons similar to those described above. The comparison component 506 communicates the differences between the positions of the robotic arms 126 and the data to the quality score component 508.

The comparison component 506 is also configured to adjust the values corresponding to positions of the robotic arms 126 and the angles for the joints of the robotic arms 126 for comparison against the data based on patient-specific characteristics. When patients of different size are operated on in system 100, the location of a particular procedure will vary relative to the table 132 based on the patient size. When comparing a procedure on a patient with a low body mass index (BMI) versus a high BMI, the location of the procedure relative to the table 132 may be nearer to or further from the upper surface of the table 132. Adjusting the robotic arm positions for patient-specific characteristics allows direct comparison of the position data for a current procedure to data from previous procedures. In one example, the robotic arm positions or the data may be adjusted or normalized based on the BMI or height and weight of a patient. In some instances, the positions, including the joint angles, positions, and locations of the robotic arms may be normalized by patient data such as BMI while the data from database 102 may likewise be normalized by the patient data for direct comparison throughout the procedure.

The comparison component 506 may compare the positions of the robotic arms 126 to the database 102 on a joint by joint or linkage by linkage basis, comparing the position and location of each joint or linkage of the robotic arms 126 to the data from the database 102 representing previous procedures. In some instances, the comparison component 506 may compare the positions and locations of each based on solely the position of the endpoint of the robotic arms 126 rather than the full length or position of each point along the length of the robotic arms 126. Additionally, the comparison component 506 may be configured to make the comparison explicitly based on the absolute position of the joints or end points of the robotic arms 126, or in some instances, the comparison component 506 may make the comparisons described herein based on averages or statistical comparisons, such as how the present normalized position of the robotic arms 126 compares to the average of normalized positions of the robotic arms 126 as represented in the database 102. In some examples, the statistical comparisons may also compare the normalized position of the robotic arms 126 to the standard deviation of a number of previous data sets within database 102 representing a number of previous procedures, each normalized for comparison as described above. In these examples, the comparison component 506 may identify whether the values representing the positions, locations, and angles of the joints of the robotic arms 126 falls within or outside of a first standard deviation of the data on database 102. These comparisons and calculations may all be performed joint by joint, on the endpoint, or on any portion of the robotic arms 126.

Turning now to the quality score component 508, the quality score component 508 is configured to determine a position quality score based on the difference determined by the comparison component 506. The quality score component 508 is configured to provide a numerical score, such as between 1 and 100, indicating the adherence of the present position of the robotic arms 126 compared to the data as compared by the comparison component 506 including any of the statistical comparisons, such as whether the values for the positions, locations, and angles of the joints are within one, two, or three standard deviations of the data within database 102. In some examples, the score may be presented in a manner besides numerical such as with a color bar, with green indicating close adherence and red indicating deviation. The score may also be accompanied with a notification provided to the user, the notification displayed at the display 118 or through a notification device and indicating a manner in which to improve the quality position score of the robotic arms 126, such as by moving a particular joint or joints to certain positions.

In some examples, the quality score component 508 may also be configured to determine an overall position quality score as well as a position quality score for each robotic arm 126. The overall position quality score may be based on an average of position quality scores for each robotic arm 126 or may be based on a weighted average, with the position quality score of more critical tools, such as a cutting tool or primary tool in a procedure, factoring more into the overall position quality score.

The display component 504 is configured to provide the position quality score from the quality score component 508 to the display 118. In some examples, the display component may be configured to output instructions, suggestions, or notifications instructing a user how to adjust the position of one or more robotic arms 126 and thereby improve the position quality score.

FIG. 6 illustrates a display 600, which is an example of the display 118, that includes user interface 602 for presenting surgical robot configuration information, according to at least one example. The user interface 602 may be presented on the display 118 of the surgical console 112 as described above, or may also be a separate display of the surgical procedure displaying video including the image data from the camera 130. In the display 600, a first robotic arm 604 and a second robotic arm 606 are visible within the field of view of the camera 130, as represented by the extents of the display 600.

Each of the first robotic arm 604 and the second robotic arm 606 are displayed on the display 600. The user interface 602 includes a first graphical interface element 608 that overlaps or aligns with the first robotic arm 604 and a second graphical interface element 610 that overlaps or aligns with the second robotic arm 606, as displayed on the display 600. The first graphical interface element 608 and the second graphical interface element 610 are shown as shapes with an outline that nearly matches the perimeter of the display of the robotic arms 604 and 606. The graphical interface elements 608 and 610 may be a color which overlaps the robotic arms to color each of the robotic arms 604 and 606 based on the unique color identified by the setup module 106 as described herein. For example, the first graphical interface element 608 may be a blue shape which overlaps the first robotic arm 604, which is associated with a blue color-configurable light attached to the first robotic arm 604.

In some examples, the first and second graphical interface elements 608 and 610 may include labels or boxes of text identifying each robotic arm 604 and 606 with their associated unique color for identification purposes. In some additional examples, the colors associated with the robotic arms may be adjusted based on an active or passive robotic arm, based on which robotic arm is currently controlled by the surgical console. For instance, the presently active robotic arm may be green while the remaining robotic arms may be red, indicating inactivity.

In the upper corner of the display 600 boxes 612, 614, and 616 are displayed including position quality scores for the robotic arms 604 and 606 as well as an overall position quality score. In the first graphical box 612 the position quality score for the first robotic arm 604 is displayed as computed by the setup module 106 described above. In the second graphical box 614 and the third graphical box 616 are displayed the position quality score for the second robotic arm 606 and the overall position quality score. In some examples, an additional box (not shown) may provide instructions for adjusting the robotic arm positions of robotic arms 604 and 606 to improve the position quality scores. For example, text in the additional box may instruct a user to adjust a third joint and a fourth joint of the first robotic arm 604 to improve the position quality score of the first robotic arm 604.

FIGS. 7-9 illustrate example flow diagrams showing processes 700, 800, and 900, according to at least a few examples. These processes, and any other processes described herein, are illustrated as logical flow diagrams, each operation of which represents a sequence of operations that can be implemented in hardware, computer instructions, or a combination thereof. In the context of computer instructions, the operations may represent computer-executable instructions stored on one or more non-transitory computer-readable storage media that, when executed by one or more processors, perform the recited operations. Generally, computer-executable instructions include routines, programs, objects, components, data structures and the like that perform particular functions or implement particular data types. The order in which the operations are described is not intended to be construed as a limitation, and any number of the described operations can be combined in any order and/or in parallel to implement the processes.

Additionally, some, any, or all of the processes described herein may be performed under the control of one or more computer systems configured with specific executable instructions and may be implemented as code (e.g., executable instructions, one or more computer programs, or one or more applications) executing collectively on one or more processors, by hardware, or combinations thereof. As noted above, the code may be stored on a non-transitory computer readable storage medium, for example, in the form of a computer program including a plurality of instructions executable by one or more processors.

Turning now to FIG. 7, FIG. 7 illustrates an example flow chart depicting a process 700 for configuring a surgical device 114, according to at least one example. The process 700 is performed by the setup module 106 (FIG. 1) executed within the computing device 104. The process 700 in particular corresponds to initially configuring or setting up a surgical device 114 with surgical tools 128 and positioning the robotic arms 126 in preparation for a procedure.

The process 700 begins at block 702 by the computing device 104 receiving image data 134 from the camera 130. In some examples, the camera 130 is an endoscope connected to an end of a robotic arm 126D to provide an up close and real-time view of the surgical area. The image data 134 may include one or more robotic arms 126 within the field of view of the camera 130.

At block 704, the process 700 includes the computing device 104 determining a first color for the first robotic arm 126A. The color may be determined based on user preference, such as a surgeon preference for the first robotic arm 126A being associated with the color blue. In some examples, the color is determined based on data the computing device receives from a database 102 of previous procedures performed using the surgical device 114. For instance, it may be common practice or standardized for the first robotic arm to always have a particular color. In some examples, the color may be determined based on the surgical tool 128A connected to the first robotic arm 126A. In these examples, the first robotic arm 126A may have a grasper or other surgical tool 128A connected to the end of the first robotic arm 126A and the color selected may reflect the surgical tool selection, with a color such as green associated with a robotic arm 126 having a grasper affixed to the end thereof.

At block 706, the process 700 includes the computing device 104 determining a second color for the second robotic arm 126B. The second color is determined based on similar parameters to the first color, though is selected to be unique with respect to the first color so as to be readily identifiable as different from the first color. In surgical devices 114 including more than two robotic arms 126, the process 700 may include the computing device 104 further determining additional unique colors for each robotic arm 126. Additionally, in some examples, the steps performed at blocks 704 and 706 may include the computing device 104 receiving an input from a user identifying a color for the first color and a color for the second color, as a user may independently select colors without relying on the database 102.

At block 708, the process 700 includes the computing device 104 causing a first color-configurable light connected to the first robotic arm 126A to emit the first color of light. The first color-configurable light may be light 116A of FIG. 2. The color-configurable light may be a light-emitting diode (LED) capable of producing light of different colors, or any other light source capable of producing light in a plurality of colors. The color-configurable light is connected to the first robotic arm 126A in such a manner that it is readily visible during setup as well as operation of the first robotic arm 126A. The light 116A may be connected at or near a joint of the first robotic arm 126A or may be positioned along a length of the first robotic arm 126A.

At block 710, the process 700 includes the computing device 104 causing a second color-configurable light connected to the second robotic arm 126B to emit the second color of light as determined at block 706. The second color-configurable light may be light 116B of FIG. 2 and be the same type of light source as described above with respect to light 116A. In some examples where the surgical device 114 includes additional robotic arms 126, process 700 may include additional steps causing color-configurable lights on each of the robotic arms 126 to emit a unique color as described above.

At block 712, the process 700 includes the computing device 104 generating a first graphical interface element and a second graphical interface element. The first and second graphical interface elements are generated based on the first color and the second color determined and associated with the first robotic arm 126A and the second robotic arm 126B. The graphical interface elements may be the graphical interface elements described in FIG. 6 above. In some instances, the color-configurable light 116 may not be visible on the display 118, so it may be otherwise represented on the display 118. The graphical interface elements are configured to identify the robotic arms within the display 118 using the first color and the second color. This block may also include identifying, using object recognition techniques known to those in the art to identify the robotic arms and thereby associate the first graphical interface element with the first robotic arm using the first color and the second graphical interface element with the second robotic arm using the second color.

At block 714, the process 700 includes the computing device causing the image data to be displayed on the display 118 at the surgical console 112. This may include causing a series of images, such as a video feed or sequence of images to be displayed in real-time for the user to view the feed of image data 134 from the camera 130. Further, at block 716, the process 700 includes the computing device causing the first and second graphical interface elements to be displayed on the display 118 with the image data 134. In some instances this may include overlapping the image data with a graphical interface element which causes the first robotic arm to appear with the first color and the second robotic arm to appear on the display with the second color.

In some examples, process 700 may include additional processes performed by the computing device, such as accessing procedure data and providing an instruction to a user describing which surgical tool 128 should be attached to each of the robotic arms 126. This may include the computing device 104 accessing database 102 and determining a configuration of the robotic arms 126 including surgical tools 128 attached thereto based on previous procedures, standard accepted procedures, surgeon preferences, or other parameter. This may be performed as part of blocks 704 and 706 or may be an entirely separate procedure.

FIG. 8 illustrates an example flow chart depicting a process 800 for determining a position quality score, according to at least one example. The process 800 is performed by the quality module 108 (FIG. 1) executed by the computing device 104. The process 800 in particular corresponds to generating a position quality score in preparation for or during a procedure using a surgical device 114.

The process 800 begins at block 802 by the computing device 104 receiving kinematic data for the robotic arms 126. The kinematic data may be the arm data 140 of FIG. 2 in some examples. The kinematic data comprises position data describing the positioning of the joints and linkages of the robotic arms 126.

At block 804, the process 800 includes the computing device 104 receiving position data from a database 102. The position data defines kinematic data describing the positions of the robotic arms at the beginning of and during a surgical procedure. The position data may be sorted according to procedure type, and may in some instances be sorted by surgeon for surgeon specific preferences. Additionally, the position data may be adjusted by patient specific parameters such as BMI as described above with respect to the position quality module of FIG. 5.

At block 806, the process 800 includes the computing device 104 comparing the kinematic data of the robotic arms 126 to the position data received from the database 102 in block 804 including the comparison described above with respect to FIG. 5. The comparison performed in block 806 includes the procedures and steps performed by the comparison component 506 of the quality module 500. The comparison component 506 compares the arm data 140 or the kinematic data to the position data. The comparison component 506 may compare the position of each robotic arm 126 joint by joint against the position data. The comparison component 506 communicates the differences between the positions of the robotic arms 126 and the data to the quality score component 508 for further process steps at block 808.

At block 808, the process 800 includes the computing device 104 determining position quality scores for the robotic arms 126 as described above with respect to the quality score component 508 of FIG. 5. In particular, the quality score component 508 determines a position quality score based on the difference determined by the comparison component 506 at block 806. The quality score component 508 provides a numerical score, such as between 1 and 100, indicating the adherence of the present position of the robotic arms 126 compared to the position data as compared by the comparison component 506 in block 806.

In some examples, block 808 involves the computing device 104 determining an overall position quality score as well as a position quality score for each robotic arm 126. The overall position quality score may be based on an average of position quality scores for each robotic arm 126 or other measures as described above.

At block 810, the process 800 includes the computing device 104 providing the position quality score for presentation at the display 118. The position quality score may be provided on the display in addition to image data from the camera 130 or may be displayed on a separate display. The position quality score may also be accompanied with instructions or text notifying the user of a manner in which to improve the quality position score of the robotic arms 126, such as by moving a particular joint or joints to certain positions. Additionally, in some examples, the position quality score may update throughout the procedure and provide a warning, such as an audible or visual notification when the arm data differs from the position data or the position quality score decreases during a procedure. Such a warning to a user may notify them that the surgeon has deviated from prior procedure or accepted procedures for a particular procedure.

FIG. 9 illustrates an example flow chart depicting a process 900 for configuring a surgical device 114 and determining a position quality score for the configuration, according to at least one example. The process 900 is performed by the computing device 104 including the position quality module and the setup module described above with respect to FIGS. 4 and 5. The process 900 in particular corresponds to setting up a surgical device 114 and determining a position quality score for the surgical device 114 during setup and performance of a procedure.

The process 900 begins at block 902 by the computing device 104 receiving image data 134 from a camera 130. This may include the same process as occurs in block 702 of FIG. 7. Next, at block 904 and 906, the computing device 104 assigns a first color and a second color in the same manner described in blocks 704 and 706 of FIG. 7. At blocks 908 and 910, the computing device 104 causes color-configurable lights 116 connected to the robotic arms 126 to emit the first color and the second color, just as in blocks 708 and 710 of FIG. 7.

At blocks 912 through 918, process 900 involves steps performed substantially as described above with respect to blocks 802 through 808 of FIG. 8. Finally, at block 920, process 900 involves the computing device 104 providing the position quality scores at the display 118. Additionally, a first and a second graphical interface element, identical to that described in blocks 710 and 712 of FIG. 7 may be generated by the setup module 106. Block 920 may further include the computing device 104 both generating and providing the first and second graphical interface elements for display at the display 118 along with the image data 134 as described above.

While the present subject matter has been described in detail with respect to specific embodiments thereof, it will be appreciated that those skilled in the art, upon attaining an understanding of the foregoing may readily produce alterations to, variations of, and equivalents to such embodiments. Accordingly, it should be understood that the present disclosure has been presented for purposes of example rather than limitation, and does not preclude inclusion of such modifications, variations, and/or additions to the present subject matter as would be readily apparent to one of ordinary skill in the art. Indeed, the methods and systems described herein may be embodied in a variety of other forms; furthermore, various omissions, substitutions and changes in the form of the methods and systems described herein may be made without departing from the spirit of the present disclosure. The accompanying claims and their equivalents are intended to cover such forms or modifications as would fall within the scope and spirit of the present disclosure.

Unless specifically stated otherwise, it is appreciated that throughout this specification discussions utilizing terms such as “processing,” “computing,” “calculating,” “determining,” and “identifying” or the like refer to actions or processes of a computing device, such as one or more computers or a similar electronic computing device or devices, that manipulate or transform data represented as physical electronic or magnetic quantities within memories, registers, or other information storage devices, transmission devices, or display devices of the computing platform.

The system or systems discussed herein are not limited to any particular hardware architecture or configuration. A computing device can include any suitable arrangement of components that provide a result conditioned on one or more inputs. Suitable computing devices include multipurpose microprocessor-based computing systems accessing stored software that programs or configures the computing system from a general purpose computing apparatus to a specialized computing apparatus implementing one or more embodiments of the present subject matter. Any suitable programming, scripting, or other type of language or combinations of languages may be used to implement the teachings contained herein in software to be used in programming or configuring a computing device.

Conditional language used herein, such as, among others, “can,” “could,” “might,” “may,” “e.g.,” and the like, unless specifically stated otherwise, or otherwise understood within the context as used, is generally intended to convey that certain examples include, while other examples do not include, certain features, elements, and/or steps. Thus, such conditional language is not generally intended to imply that features, elements and/or steps are in any way required for one or more examples or that one or more examples necessarily include logic for deciding, with or without author input or prompting, whether these features, elements and/or steps are included or are to be performed in any particular example.

The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. The use of “adapted to” or “configured to” herein is meant as open and inclusive language that does not foreclose devices adapted to or configured to perform additional tasks or steps. Additionally, the use of “based on” is meant to be open and inclusive, in that a process, step, calculation, or other action “based on” one or more recited conditions or values may, in practice, be based on additional conditions or values beyond those recited. Similarly, the use of “based at least in part on” is meant to be open and inclusive, in that a process, step, calculation, or other action “based at least in part on” one or more recited conditions or values may, in practice, be based on additional conditions or values beyond those recited. Headings, lists, and numbering included herein are for ease of explanation only and are not meant to be limiting.

The various features and processes described above may be used independently of one another, or may be combined in various ways. All possible combinations and sub-combinations are intended to fall within the scope of the present disclosure. In addition, certain method or process blocks may be omitted in some implementations. The methods and processes described herein are also not limited to any particular sequence, and the blocks or states relating thereto can be performed in other sequences that are appropriate. For example, described blocks or states may be performed in an order other than that specifically disclosed, or multiple blocks or states may be combined in a single block or state. The example blocks or states may be performed in series, in parallel, or in some other manner. Blocks or states may be added to or removed from the disclosed examples. Similarly, the example systems and components described herein may be configured differently than described. For example, elements may be added to, removed from, or rearranged compared to the disclosed examples.

What is claimed is:

1. A robotic surgical system, comprising:
a plurality of robotic arms, at least two robotic arms of the plurality of robotic arms each configured to couple with an interchangeable surgical tool at an end thereof and each of the plurality of robotic arms having a corresponding light emitting device;
a camera positionable to capture images of the at least two robotic arms; and
one or more processors; and
one or more non-transitory computer-readable media comprising computer-executable instructions that, when executed by the one or more processors, cause the one or more processors to:
receive, from the camera, an image that depicts the at least two robotic arms;
provide a representation of the image for presentation at a display;
determine a first color of a first robotic arm of the at least two robotic arms based on a first arm characteristic associated with the first robotic arm;
determine a second color of a second robotic arm of the at least two robotic arms based on a second arm characteristic associated with the second robotic arm;
cause the light emitting device corresponding to the first robotic arm to emit the first color;
cause the light emitting device corresponding to the second robotic arm to emit the second color; and
provide a first graphical interface element and a second graphical interface element for presentation at the display together with the image, the first graphical interface element associating the first robotic arm with the first color and the second graphical interface element associating the second robotic arm with the second color.

2. The robotic surgical system of claim 1, wherein the first arm characteristic comprises a first interchangeable tool selection and the second arm characteristic comprises a second interchangeable tool selection.

3. The robotic surgical system of claim 1, wherein the first arm characteristic is based on robotic surgery data and the second arm characteristic is based on the robotic surgery data, the robotic surgery data corresponding to configurations of the plurality of robotic arms for a surgical procedure.

4. The robotic surgical system of claim 3, wherein the robotic surgery data comprises a surgeon preference.

5. The robotic surgical system of claim 3, wherein the robotic surgery data comprises a tool assignment for the plurality of robotic arms.

6. The robotic surgical system of claim 1, wherein the first graphical interface element comprises a portion of the image including the first robotic arm and wherein associating the first robotic arm with the first color comprises adjusting a color of the portion of the image based on the first color.

7. The robotic surgical system of claim 6, wherein adjusting the color of the portion of the image based on the first color comprises identifying a portion of the image comprising the first arm and applying the first color to the portion of the image.

8. The robotic surgical system of claim 1, wherein the first arm characteristic identifies a first one of the plurality of robotic arms and the second arm characteristic identifies a second one of the plurality of robotic arms.

9. A computer-implemented method, comprising:
receiving an image from a camera positioned to view at least a portion of a robotic surgical system, the image depicting at least two robotic arms of the robotic surgical system;
determining a first color for a first robotic arm of the at least two robotic arms based on a first arm characteristic associated with the first robotic arm;
determining a second color for a second robotic arm of the at least two robotic arms based on a second arm characteristic associated with the second robotic arm;
causing a first light emitting device connected to the first robotic arm to emit the first color;
causing a second light emitting device connected to the second robotic arm to emit the second color.

10. The computer-implemented method of claim 9, wherein the first arm characteristic comprises a first tool selection and the second arm characteristic comprises a second tool selection.

11. The computer-implemented method of claim 9, wherein the first arm characteristic is based on surgical data and the second arm characteristic is based on the surgical data, the surgical data corresponding to configurations of the at least two robotic arms for a surgical procedure.

12. The computer-implemented method of claim 11, wherein the surgery data comprises a surgeon preference.

13. The computer-implemented method of claim 11, wherein the surgery data comprises a tool assignment for the at least two robotic arms.

14. The computer-implemented method of claim 9, further comprising:
generating a first graphical interface element and a second graphical interface element, the first graphical interface element identifying the first robotic arm using the first color and the second graphical interface element identifying the second robotic arm using the second color;
causing the image to be displayed; and
causing the first and second graphical interface elements to be displayed with the image.

15. The computer-implemented method of claim 14, wherein the first graphical interface element comprises a portion of the image including the first robotic arm and wherein associating the first robotic arm with the first color comprises adjusting a color of the portion of the image based on the first color.

16. The computer-implemented method of claim 9, wherein the first arm characteristic identifies a first one of the at least two robotic arms based on a first tool assignment and the second arm characteristic identifies a second one of the at least two robotic arms based on a second tool assignment.

17. A robotic surgical system, comprising:
a plurality of robotic arms, at least two robotic arms of the plurality of robotic arms each configured to couple with a surgical tool at an end thereof;
a camera positionable to capture images of the at least two robotic arms;
one or more processors; and
one or more non-transitory computer-readable media comprising computer-executable instructions that, when executed by the one or more processors, cause the one or more processors to:
receive kinematic data for the at least two robotic arms, the kinematic data corresponding to a position and an orientation of each of the at least two robotic arms;
access orientation data corresponding to a plurality of reference positions and orientations for the plurality of robotic arms, the orientation data representing positions and orientations of robotic arms of the robotic surgery systems during a surgical procedure;
comparing the kinematic data and the orientation data to identify one or more orientation differences for the at least two robotic arms;
determine position quality scores for the at least two robotic arms based on the one or more orientation differences; and
provide, for presentation at a display, the position quality scores for the at least two robotic arms.

18. The robotic surgery system of claim 17, wherein the one or more non-transitory computer-readable media further comprise additional computer-executable instructions that, when executed by the one or more processors, cause the one or more processors to:
determine an overall position quality score for the plurality of robotic arms based on the position quality scores for the at least two robotic arms; and
provide, for presentation at the display, the overall position quality score.

19. The robotic surgery system of claim 17, wherein the position quality scores for the at least two robotic arms are further based on a comparison of the kinematic data and a center of a range of motion of each of the at least two robotic arms.

20. The robotic surgery system of claim 17, wherein the position quality scores for the at least two robotic arms are further based on a distance between ends of the at least two robotic arms.

21. The robotic surgery system of claim 17, wherein the one or more non-transitory computer-readable media further comprise additional computer-executable instructions that, when executed by the one or more processors, cause the one or more processors to:
generate a recommendation for adjusting the orientations of the at least two robotic arms; and
provide instructions to a user to adjust the orientations of the at least two robotic arms based on the recommendation.

22. The robotic surgery system of claim 17, wherein the one or more non-transitory computer-readable media further comprise additional computer-executable instructions that, when executed by the one or more processors, cause the one or more processors to:
simulate future orientations of the at least two robotic arms based on the orientation data; and
identify, based on the future orientations, instances of potential collision between the at least two robotic arms during the surgical procedure.

23. The robotic surgery system of claim 22, wherein the one or more non-transitory computer-readable media further comprise additional computer-executable instructions, that when executed by the one or more processors, cause the one or more processors to:
provide instructions to a user for moving the at least two robotic arms prior to performing the surgical procedure based on the instances of potential collision between the at least two robotic arms during the surgical procedure to avoid the instances of potential collision.

24. The robotic surgery system of claim 17, wherein comparing the kinematic data and the orientation data comprises adjusting the orientation data based on a patient characteristic.

25. A computer-implemented method, comprising:
receiving kinematic data for at least two robotic arms of a robotic surgical system, the kinematic data corresponding to a position and an orientation of each of the at least two robotic arms;
accessing orientation data corresponding to a plurality of reference positions and orientations for the at least two robotic arms based on previous surgical procedures performed by robotic surgical systems, the orientation data representing positions and orientations of robotic arms of the robotic surgical systems during the surgical procedure;
comparing the kinematic data and the orientation data to identify one or more orientation differences for the at least two robotic arms;
determining position quality scores for the at least two robotic arms based on the one or more orientation differences; and
providing, for presentation at a display, the position quality scores for the at least two robotic arms.

26. The computer-implemented method of claim 25, further comprising:
determining an overall position quality score for the at least two robotic arms based on the position quality scores for the at least two robotic arms; and
providing, for presentation at the display, the overall position quality score.

27. The computer-implemented method of claim 25, wherein the position quality scores for the at least two robotic arms are further based on a comparison of the kinematic data and a center of a range of motion of each of the at least two robotic arms.

28. The computer-implemented method of claim 25, wherein the position quality scores for the at least two robotic arms are further based on a distance between ends of the at least two robotic arms.

29. The computer-implemented method of claim 25, further comprising:
generating a recommendation for adjusting the orientations of the at least two robotic arms; and
instructing adjustment of the orientations of the at least two robotic arms based on the recommendation.

30. The computer-implemented method of claim 25, further comprising:
simulating future orientations of the at least two robotic arms based on the orientation data; and
identifying, via the future orientations, instances of potential collision between the at least two robotic arms during the surgical procedure.

31. The computer-implemented method of claim 30, further comprising:
providing instructions to a user to move the at least two robotic arms prior to performing the surgical procedure based on the instances of potential collision between the at least two robotic arms during the surgical procedure to avoid the instances of potential collision.

32. A computer-implemented method, comprising:
receiving an image from a camera positioned to view at least a portion of a robotic surgical system, the image depicting at least two robotic arms of the robotic surgical system;
determining a first color for a first robotic arm of the at least two robotic arms based on a first arm characteristic associated with the first robotic arm;
determining a second color for a second robotic arm of the at least two robotic arms based on a second arm characteristic associated with the second robotic arm;
causing a first light emitting device connected to the first robotic arm to emit the first color;
causing a second light emitting device connected to the second robotic arm to emit the second color;
receiving kinematic data for the at least two robotic arms, the kinematic data corresponding to a position and an orientation of each of the at least two robotic surgery;
accessing orientation data corresponding to a plurality of reference positions and orientations for the at least two robotic arms based on previous surgical procedures performed by robotic surgical systems, the orientation data representing positions and orientations of robotic arms of the robotic surgical systems during the surgical procedure;
comparing the kinematic data and the orientation data to identify one or more orientation differences for the at least two robotic arms;
determining position quality scores for the at least two robotic arms based on the one or more orientation differences;
generating a first graphical interface element and a second graphical interface element, the first graphical interface element identifying the first robotic arm using the first color and including a first position quality score of the position quality scores corresponding to the first robotic arm, the second graphical interface element identifying the second robotic arm using the second color and including a second position quality score of the position quality scores corresponding to the second robotic arm;
causing the image to be displayed; and
causing the first and second graphical interface elements to be displayed with the image.

33. The computer-implemented method of claim 32, further comprising:
accessing previous surgery data describing orientation of the at least two robotic arms;
identifying, via the previous surgery data, instances of potential collision between the at least two robotic arms during the surgical procedure; and
providing, at a display, a notification of the instances of potential collision.

34. The computer-implemented method of claim 32, further comprising:
simulating future orientations of the at least two robotic arms based on the orientation data;
identifying, via the future orientations, instances of potential collision between the at least two robotic arms during the surgical procedure; and
providing, at a display, a notification of the instances of potential collision.

35. The computer-implemented method of claim 32, further comprising:
simulating future orientations of the at least two robotic arms based on the orientation data;
identifying, via the future orientations, instances of potential collision between the at least two robotic arms during the surgical procedure; and
instructing movement of the at least two robotic arms to prevent the instances of potential collision.
